(19) United States
(12) Patent Application Publication
(10) Pub. No.: US 2011/0223314 A1
(43) Pub. Date: Sep. 15, 2011

(54) EFFICIENT MICROENCAPSULATION

(76) Inventors: Xiaoxiao ZHANG, Honolulu, HI (US); David Garmire, Honolulu, HI (US); Aaron Ohta, Honolulu, HI (US)

(21) Appl. No.: 13/045,244

(22) Filed: Mar. 10, 2011

Related U.S. Application Data
(60) Provisional application No. 61/339,942, filed on Mar. 10, 2010.

(51) Int. Cl. B05D 7/00; B05C 3/02; B05C 11/00; B05D 1/18; B05D 3/02; B05D 3/06; B82Y 5/00

(52) U.S. Cl. 427/2.1; 118/400; 118/642; 118/58; 427/212; 427/512; 977/904

(57) ABSTRACT

A device and method for generating microcapsules employs an inertial-focusing channel for introducing particles dispersed in a prepolymer suspension fluid, a droplet-generating junction for introducing oil evenly onto the flow of particles to create separated droplets of prepolymer suspension fluid encapsulating respective particles in a streamline flow, and a polymerization section for exposing the droplets to UV light or heat to cause polymerization of a polymer coating on separate microcapsules each containing a respective particle. Preferred suspension fluids may be aqueous solution of poly(ethylene-glycol)-diacrylate (PEGDA), or poly(N-isopropyl-acryalmide) (PNIPAAM). The preferred device may employ a curved or linear inertial-focusing microchannel. Functional tags and/or handles may be added to the microcapsules allowing easy detection, measurement and handling of the microcapsules.

This U.S. patent application claims the priority of U.S. Provisional patent application 61/339,942 filed on Mar. 10, 2010, by the same inventors, and of the same title.

### TECHNICAL FIELD

The presently disclosed invention relates generally to microencapsulation, and particularly to methods of operation and devices for continuously generating monodispersed microcapsules of controllable size and content of bioparticles, cells, or groups of cells.

### BACKGROUND OF THE INVENTION

Microencapsulation is the process of surrounding tiny particles or droplets with a uniform coating or wall, thereby generating structures having remarkable properties useful in a variety of applications, including material sciences, pharmaceuticals, biotechnology and cell-based treatments. In many of these applications, microencapsulation provides a means of protecting or separating sensitive contents that one wishes to manipulate or monitor (sense) within a given environment, often in minutes quantities. For example, the idea of using microencapsulation to maintain and protect cellular machinery has long been a longstanding goal in the field of cellular biology and medicine.

One promising application of microencapsulation is in cellular therapeutics. The field of cellular therapeutics offers a modality for treating hormone, enzyme, and factor-related diseases. It involves the use of cells that are transplanted or injected in patients. The cells function as in vivo “factories,” continually producing therapeutic agents. Cell-based treatments can be more effective than drug or protein-based treatments which are one-time delivery methods. Furthermore, drug treatment concerns are minimized, such as overdosing due to the rupture of delivery capsules. A major issue with cellular therapeutics is the protection of the implanted cells from the patient's immune response.

Another emerging area that has drawn increasing research interest is the study of cell behavior at the single-cell level. For this purpose, much work has been done to create cell arrays for carrying out single-cell bioassays, including measurement of single-cell respiration rates, drug screening down to single-cell levels, viability studies with micro-environmental control, monitoring of cellular gene expression, and intercellular interactions. This requires the ability to manipulate and tag cells with single-cell resolution and high throughput without interfering with cellular functions. Currently, manipulation and tagging of cells is achieved by adding functional elements, like proteins, which bind to the surface or are dispersed internally within a cell. These proteins may contain fluorescent tags, micro-acoustic markers, and other functionalized elements. The main issue with these approaches is the often unpredictable nature of protein-cellular and protein-marker interactions. Significant experimentation is often required to prove the desired properties are present in the tags.

Cell encapsulation is a technology that uses semi-permeable microcapsules for the protection of transplanted cells, while allowing the exchange of nutrients and waste, and the release of therapeutic agents. Encapsulation for cellular therapeutics is a promising alternative approach for the treatment of numerous diseases including diabetes, cancer, central nervous system diseases, and endocrinological disorders. Moreover, encapsulation of single-cells can be a great tool for biologists to conduct single-cell level bioassays, including the monitoring of cellular gene expression, drug screening at single-cell levels, viability studies under microenvironmental control, monitoring of intercellular interactions, and measurement of single-cell respiration rates.

Since cell encapsulation was first proposed by T. M. S. Chang, Semipermeable Microcapsules, 146 Science 524, 524-25 (1964), a significant amount of research has been done to bring microencapsulation both biologically and technologically closer to clinical applications. However, microencapsulation still remains largely an “in-lab” procedure, largely due to the lack of a standardized technology that is capable of producing uniform capsules with repeatability both within and between batches in terms of size and number of encapsulated particles.

The most common methods of microencapsulation are droplet extrusion and emulsification. The former technique produces capsules in the millimeter-size-range, which are too large for single-cell encapsulation, while the latter method suffers from uncontrolled capsule size distribution. Furthermore, neither method has control of the number of encapsulated cells (hereinafter referred to as “occupancy”). Microfluidic technology has been employed to produce monodispersed microcapsules having diameters as small as 100 micrometers (μm), but the occupancy remains uncontrolled. As applied to cellular encapsulation, this inability to control occupancy significantly reduces the number of usable capsules and causes a large uncertainty in subsequent biological experiments, jeopardizing the reliability and repeatability of the research results. Therefore, a method and device for producing monodispersed microcapsules with controlled occupancy is needed.

### BRIEF SUMMARY OF THE INVENTION

A first aspect of the present invention is a method and device for generating microcapsules encapsulated in a polymer coating containing single or multiple cells, particles, liquids, or other matter, wherein the size and occupancy of the microcapsules may be selectively controlled. A device for generating microcapsules encapsulated in a polymer coating comprises: a microfluidic channel having an inlet for particles dispersed in a random spacing in a prepolymer suspension fluid, an outlet for exiting particles carried at a relatively even spacing in the suspension fluid, and an inertial-focusing microchannel section between the inlet and outlet having channel dimensions and shape to cause the particles to become relatively evenly spaced in a streamline flow; a droplet-generating junction at the microchannel outlet having two opposing oil channels for introducing an continuous oil phase fluid evenly on opposing sides of the flow of particles so as to create separated droplets of prepolymer suspension fluid encapsulating respective particles in the streamline flow; and a polymerization section for exposing the droplets to a physical energy/reagent causing polymerization of the prepolymer suspension fluid so as to polymerize separate prepolymer droplets each containing a controlled amount of respective particles.

The prepolymer suspension fluid is preferably an aqueous solution of a biocompatible prepolymer hydrogel with a viscosity close to that of water. Preferred fluids include an aqueous solution of poly(ethylene-glycol)-diacrylate (PEGDA), and poly(N-isopropyl-acrylamide) (PNIPAAM). The permeability and other characteristics of the polymer encapsulation may be controlled or altered, and may be selected for polymerization by exposure to UV light, heat, or other physical energy or reagent. In one embodiment, a microfluidic device containing a straight inertial-focusing microchannel is capable of encapsulating particles of about 10 μm diameter within droplets of about 60 μm diameter at a rate greater than 200 Hz.

Another aspect of the present invention is a method and compact device for generating microcapsules encapsulated in a polymer coating containing single or multiple cells, particles, liquids, or other matter, wherein particles of different sizes within a mixture may be separated and selectively encapsulated into microcapsules of controllable size and occupancy. A preferred apparatus comprises a curved (spiral) inertial-focusing microchannel, microdroplet-generating junction, and polymerization section which together provide a compact device capable of separating and microencapsulating individual particles from mixtures of particles, wherein the permeability and other characteristics of the microcapsule may be controlled or altered. The process is both high-throughput and repeatable. In one embodiment, a microfluidic device containing a curved (spiral) inertial-focusing channel with increasing radius and channel width is capable of selectively microencapsulating 10-μm-diameter and 20-μm-diameter particles from mixtures containing both particles at a rate of greater than 200 Hz.

Another aspect of the invention is a method for continuously generating microcapsules of controlled occupancy and size, wherein functional “tags” and/or “handles” may be added to the microcapsules during microencapsulation to allow easy detection and physical manipulation. The ability to add additional ingredients to microcapsules generated using the devices described herein permits incorporation of functional characteristics, such as fluorescence, magnetism, quantum dots and other features useful for manipulation, monitoring and measurement.

Other aspects, features, and advantages of the present invention will be explained in the following detailed description of embodiments thereof, having reference to the appended drawings.

### BRIEF DESCRIPTION OF THE DRAWINGS

FIG. 1 shows a plot of the theoretical yield of single-particle capsules comparing inertially-focused particles versus particles randomly distributed according to Poisson statistics.

FIG. 2a shows a side-view illustration of the process of inertial-focusing in straight or curved (spiral) microfluidic channels.

FIG. 2b shows a top-view illustration of the process of inertial-focusing in straight or curved (spiral) microfluidic channels.

FIG. 3 shows a schematic drawing of one embodiment of a microfluidic device comprising an inertial-focusing microchannel, a droplet-generating junction, and a photopolymerization section.

FIGS. 4a-4c illustrate one embodiment of the process used to fabricate the microfluidic devices of the present invention.

FIGS. 5a-5b show a schematic diagram of an experimental setup using one embodiment of a straight-channel microfluidic device.

FIG. 6a shows a plot of the estimated kinetic viscosity of a liquid mixture of poly(ethylene-glycol)-diacrylate (PEGDA) in water at 25° C.

FIGS. 7a-7c depict the results of an experiment using one embodiment of a straight-channel microfluidic device to inertially focus 10.3-μm-diameter polystyrene beads as cell simulants.

FIG. 8 illustrates a proof-of-principle study demonstrating that the droplet-generating junction and photo-polymerization sections function properly to generate microcapsules.

FIGS. 9a-9b show schematic diagrams of an experimental setup using one embodiment of a compact curved (spiral)-channel microfluidic device.

FIGS. 10a-10e illustrate the results using the curved (spiral)-channel device depicted in FIG. 9.

FIG. 11 shows a plot of the equilibrium positions of 10-μm-diameter and 20-μm-diameter polystyrene beads at the outlet of the inertial focusing section for the microfluidic device depicted in FIG. 9.

FIG. 12 illustrates the selective microencapsulation of 20 μm particles from 10 μm particles using the curved-(spiral)-channel microfluidic device depicted in FIG. 9.

FIG. 13 illustrates the process of generating microcapsules containing functional “tags” and/or “handles.”

### DETAILED DESCRIPTION OF THE INVENTION

A first aspect of the present invention is a method and device for efficiently and rapidly encapsulating cells, minute particles, liquids, and other matter, wherein the size of the microcapsules and the number of encapsulated particles can be controlled. Although some microfluidic devices are known to be capable of producing monodispersed microcapsules amenable to cell encapsulation and other applications, the particle-loading dynamics in these devices generally reduce the yield of usable capsules because the amount of particles per capsule (hereinafter referred to as the “occupancy”) varies according to Poisson statistics. As a result, single-particle encapsulation using previous methods was only attainable at low particle loading densities, such that a significant fraction of the microcapsules produced are empty. For example, the yield of usable particle-containing droplets using earlier methodologies will be less than 10% when the average number of particles per capsule is 1.1.

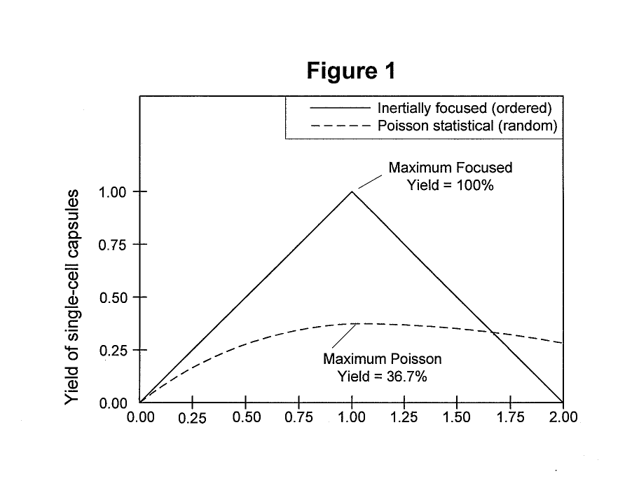

The traditional methods of microencapsulation—droplet extrusion and emulsification—are governed by Poisson statistics. FIG. 1 shows a plot of the theoretical yield of single-particle capsules comparing the traditional (Poisson) methods versus the inertial-focusing methodology employed in this invention. As shown, the Maximum Poisson Yield of single-particle capsules under Poisson statistics is limited to 36.7%, when the particles are randomly distributed. However, in the case of inertial focusing, wherein the particles form a regularly-spaced array, the Maximum Focused Yield may reach 100%. Therefore, the loading of cells and other particles into droplets (and ultimately capsules) can be made more controllable and repeatable by using inertial focusing to transform non-ordered groups of particles into regularly-spaced arrays amenable to single-particle encapsulation. Inertial focusing, which uses the inertial lift forces to focus particles into predictable spatial locations within a channel, can be achieved within microchannels.

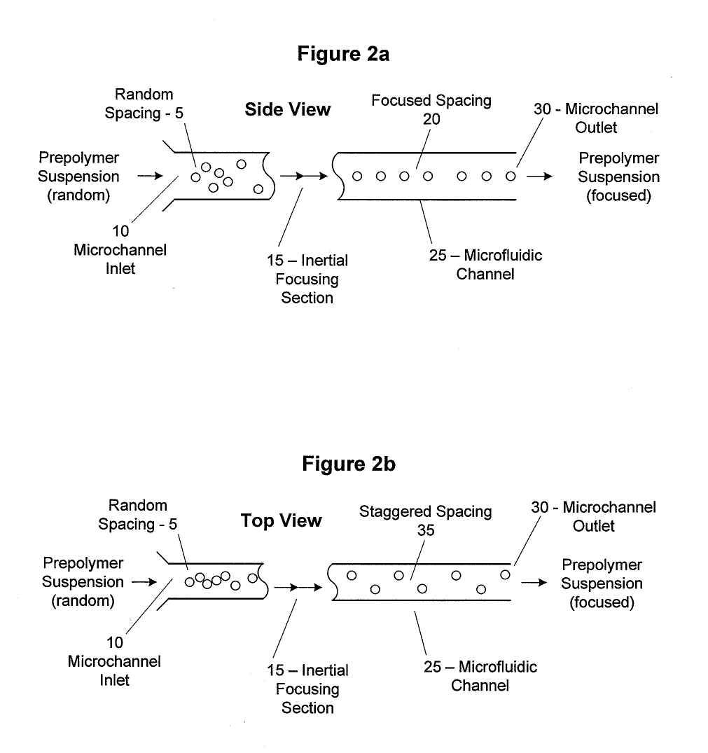

In microfluidic devices employing both straight and spiral channels, inertial focusing phenomena can be observed when the microchannel length and flow rate fulfill certain criteria. FIGS. 2a and 2b illustrate the process of inertial-focusing of particles within the microchannel of a microfluidic device. FIG. 2a is a side-view illustration of the inertial focusing an exemplary microfluidic channel 25. As shown, randomly-dispersed particles 5 are introduced into a microchannel inlet 10 of given inlet dimensions and enter an inertial-focusing microchannel section 15 having channel dimensions narrowed from that of the inlet and shaped to utilize an interplay of fluid forces attributable to the flow of the suspension fluid on the particles so as to cause the particles to become relatively evenly spaced within a streamline flow of the suspension fluid. This means that after the particles exit from the microchannel outlet 30, and are directed to a droplet-generating junction, they will pass through the junction serially, at repeatable fixed intervals—a property that is crucial in single-particle encapsulation. In certain embodiments, the shape of the microchannel produces a staggered streamline of particles. FIG. 2b is a top-view illustration of the inertial focusing the same exemplary microfluidic channel depicted in FIG. 2a. In this example, the focused particles are observed to be evenly-spaced in a staggered, planar arrangement, as opposed to the non-staggered arrangement as shown from the correspond side view (FIG. 2a).

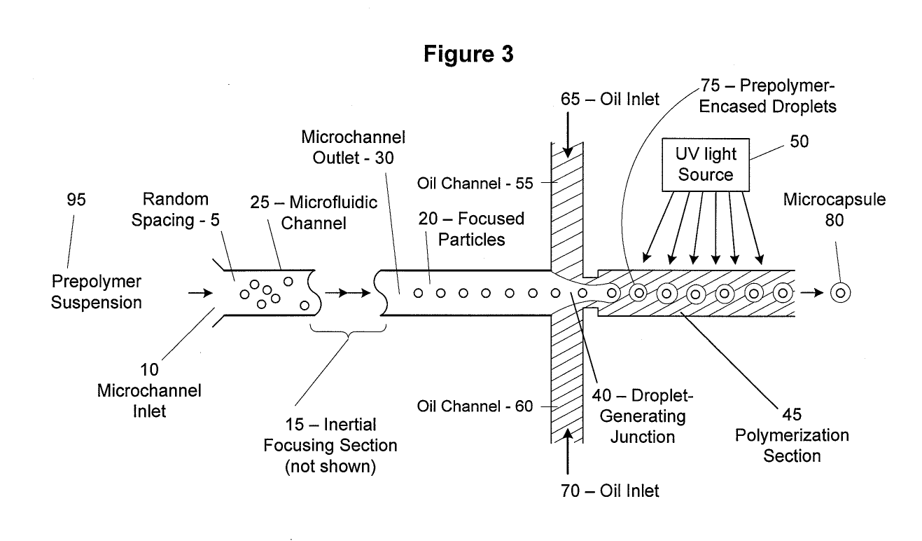

FIG. 3 shows a schematic illustration of one embodiment of the microencapsulation device of present invention. The microencapsulation devices of the present invention are comprised of an inertial focusing section 15, located immediately after the microchannel inlet 10, a droplet-generation junction 40 (hereinafter referred to as the “junction”) located after the microchannel outlet 30, and a polymerization section 45 located directly downstream of the junction 40, which may employ a photon source (e.g. UV) 50 to affect rapid polymerization. The inertial focusing section 15 may be comprised of a straight or curved (spiral) channel of defined dimensions to ensure adequate spacing of the focused particles 20. The droplet-generation junction 40 contains at least two opposing oil channels 55 and 60 allowing the introduction 65 and 70 of an oil phase and the creation of separated, prepolymer-encased droplets 75, which later form microcapsules 80 in the polymerization section 45. The design of the polymerization section 45 slows down the linear flow rate of the prepolymer-encased droplets 75 and exposes them to UV light 50, or another polymerization initiator, causing polymerization of the prepolymer layer and encapsulation to form microcapsules 80. FIG. 3b shows an exploded view of a microcapsule 80 formed using the devices of the present invention. Each singly-occupied microcapsule 80 is comprised of the polymerized capsule 85 enclosing the particle 90. The hydrogel itself is the polymer capsule, such that the entire droplet polymerizes or hardens.

One embodiment of the present invention operates as follows. First, a prepolymer suspension 95 is prepared by mixing of poly(ethylene-glycol)-diacrylate (PEGDA) with a photo-initiator and particles, or a mixture of particles, to be encapsulated. Next, the prepolymer suspension 95 is then pumped into the inlet of the microfluidic channel, which is designed so that the particles are stably self-organized before they reach the droplet-generating junction 40. At the junction 40, oil is introduced from the oil channels 55 and 60 causing the formation of an emulsion in which prepolymer-encased droplets 75 of the PEGDA mixture are formed. Finally, within the polymerization section 45 of the device, the PEGDA surrounding the droplets 75 undergoes UV-induced polymerization to form a particle-containing microcapsule 80. Single-particle encapsulation occurs when the droplets 75 are generated at the same or higher frequency than the frequency at which particles enter the junction 40. Both frequencies are controlled by the relative flow rates of the prepolymer suspension 95 (e.g., hydrogel) through the microfluidic channel 30 and the oil through the oil channels 65 and 70.

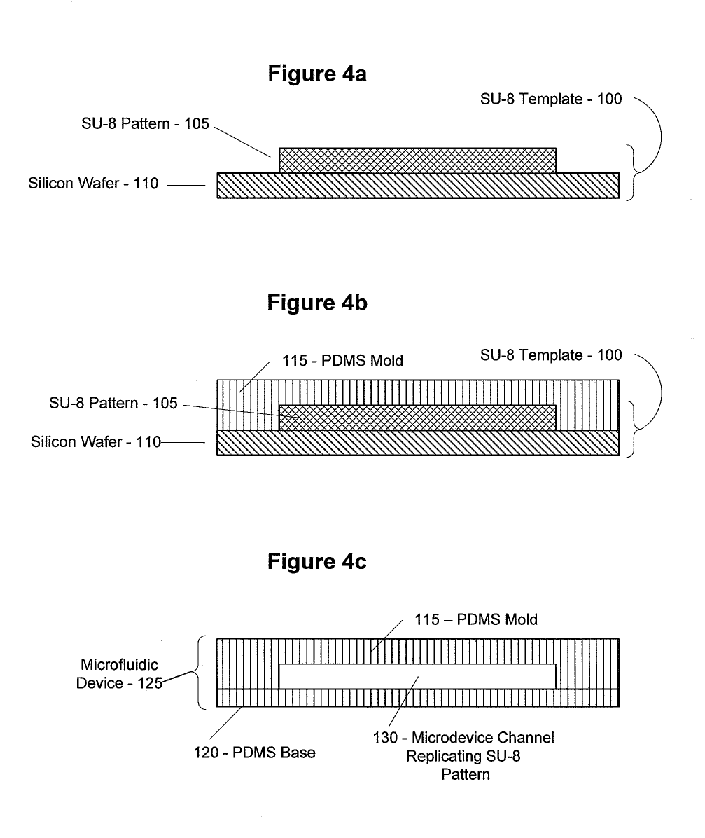

FIGS. 4a to 4c illustrate one embodiment of the process for manufacturing microfluidic devices from polydimethylsiloxane (PDMS). A standard soft lithography technique is utilized wherein SU-8 50 (MicroChem) is spin-coated at 2000 rpm for 30 seconds to create a 50 μm thick layer on a 4″ silicon wafer. FIG. 4a shows one embodiment of a SU-8 template 100 comprising an SU-8 pattern 105 deposited onto a silicon wafer 110 using standard photolithography and development techniques. A mixture of the polymer (PDMS, Sylgard 184; Dow Corning) base and crosslinker, having base-to-crosslinker ratios ranging from 8:1 to 12:1, is then poured onto the SU-8 master 110. FIG. 4b shows one embodiment of the resulting PDMS mold 115 formed onto the SU-8 pattern 105 of the SU-8 template 100. After degassing in a vacuum chamber and curing at 65° C. for about 4 hours, the PDMS mold 115 and the SU-8 template 100 are released, and holes are drilled to create inlets and outlets. A PDMS base 120 is then attached to the PDMS mold 115 to form the microfluidic device 125 with a microdevice channel 130 replicating the SU-8 pattern 105, and the device is cured at 65° C. overnight. FIG. 4c illustrates one embodiment of the microfluidic device following release from the SU-8 template 110 and attachment to a lower PDMS base 120. Rain-X™ (Rain-X original; Sopus Products) or Aquapel™ (Pittsburgh Glass Works LLC) is finally forced through the microfluidic channel to ensure that hydrophobic surfaces exist throughout the channel. Hydrophobicity can be increased by allowing the Rain-X™ or Aquapel™ to evaporate. The same bonding procedure can be used to bond a PDMS structural layer made with a 10:1 base-to-crosslinker ratio to a glass substrate that has been Piranha treated (4:1 H₂SO₄:H₂O₂).

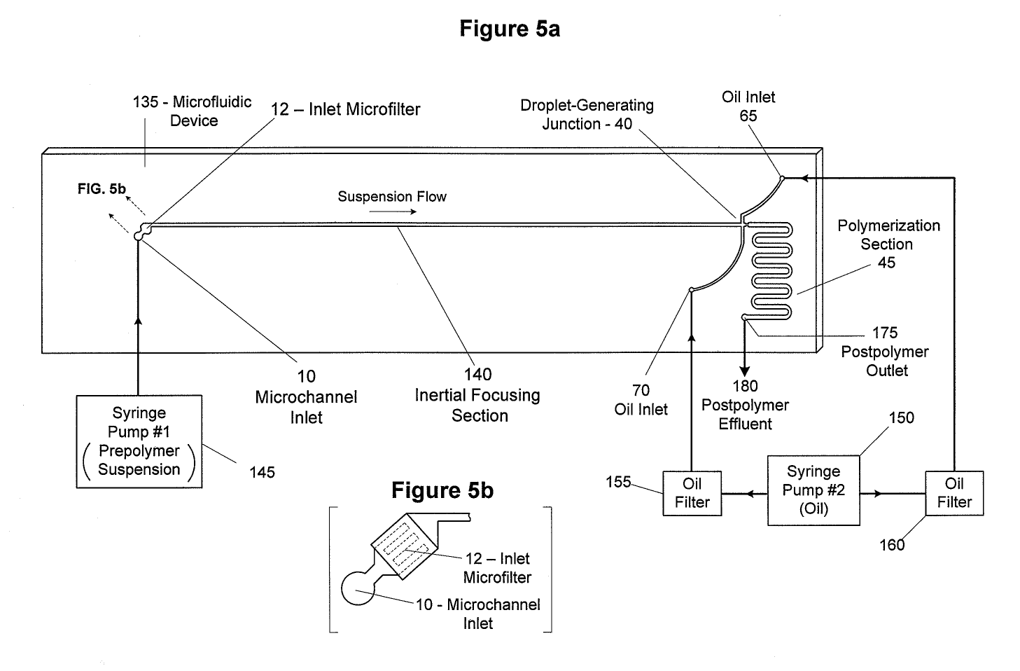

FIG. 5a illustrates the experimental setup for one embodiment of a microfluidic device 135, fabricated as described above and employing a linear-channel inertial-focusing section 140 with a rectangular cross section measuring 27 nm wide, 50 nm high and 6 cm in length. This device 135 was successfully tested using fluorescent 10.2 μm polystyrene beads to simulate cells of similar size.

In a typical experiment, a premixed and emulsified prepolymer suspension 95 is pumped into the microchannel inlet 10 and through an inlet microfilter 12 using syringe pump #1 145 (KDS-201, KD Scientific), while a fluorinert oil (FC-40, 3M) is pumped into the oil inlets 65 and 70 using syringe pump #2 150. FIG. 5b shows an exploded view of the microchannel inlet 10 and microfilter 12 sections of the microfluidic device. A set of two 0.2 nm syringe filters 155 and 160 are placed between syringe pump #2 150 and the oil inlets 65 and 70 to remove particulate impurities in the oil. As the prepolymer suspension 95 migrates along the straight channel, the polystyrene particles are focused into an evenly-spaced streamline with a well-defined lateral equilibrium position, which depends largely upon the flow rate, particle size, concentration and viscosity of the prepolymer suspension. The inertially-focused particles then flow into the droplet-generating junction 40 wherein appropriate oil/hydrogel mixing forms prepolymer-encased droplets 75 containing the polyethylene beads. The occupancy of the resulting droplets 75 is dependent, in part, upon the respective flow rates of the prepolymer suspension 95 and the oil—such that single-particle encapsulation occurs when droplets 75 are generated at the same or higher frequency than the frequency at which the beads enter the junction 40. Thus, occupancy is controlled, in part, by the respective flow rates of syringe pumps #1 145 and #2 150. The droplets 75 then enter a polymerization section 45, where polymer base undergoes photo-induced or thermally-induced polymerization to form particle-containing microcapsules 80. In one embodiment, the polymer base is PEGDA and the polymerization section 45 uses UV light to induce polymerization. In another embodiment, the polymer base is poly(N-isopropyl-acrylamide) (PNIPAAM) and the polymerization section uses heat to induce polymerization. Finally, the polymerized microcapsules 80 exit the device via a postpolymer outlet 175 and are collected within the postpolymer effluent 180.

The PDMS devices 135 were mounted on a microscope (BX45, Olympus) with a high speed camera (GE680C, Prosilica). Within the polymerization section 45, UV exposure of 365 nm at 10 mW/cm2 was generated by a UV light source (LC8, Hamamatsu). Maintaining sufficient homogeneity of the particle/prepolymer suspension 95 is necessary to ensure continuous and reliable inertial focusing both linear and curved-channel devices. For this purpose, the suspension can be constantly stirred or the density of the prepolymer solution can be adjusted to match that of the cells/particles to be encapsulated.

In order to achieve inertial focusing of particles in an aqueous solution of PEGDA, parameters such as viscosity and flow velocity of the mixture must be adjusted to maintain an appropriate Reynolds number. In fluid mechanics, the Reynolds number (Re) is a dimensionless number that gives a measure of the ratio of inertial forces to viscous forces, and consequently quantifies the relative importance of these two types of forces for given flow conditions. The Reynolds number may be expressed as:

$$Re = ρ \frac{{U}_{m} {D}_{k}}{μ}$$

where ρ is the liquid density, Um is channel velocity, Dh is the hydraulic diameter of the channel, and μ is the liquid viscosity. Inertial focusing has been demonstrated in water (μ=1 cSt at 25° C.) in microchannels under a resonable flow velocity. But the viscosity of pure PEGDA is 50.89 cSt, and to focus particles in pure PEGDA it will need flow velocity that is 50 times higher than that in pure water, which will cause device rapture. Therefore, it is necessary to dilute the PEGDA to achieve the appropriate viscosity for inertial focusing to take place at a lower flow velocity. For the experiments described herein, the viscosity of the mixture of PEGDA in water at different mixing ratios was estimated by calculating the viscosity blending index (VBI) of aqueous PEGDA using Refutas equation as:

VBI≈14.534×ln[ln(ν+0.8)]+10.975

where ν is the kinetic viscosity of the component. The VBI of the mixture is calculated as:

$${VBI}_{blend} = {\sum }_{i} {W}_{i} {VBI}_{i}$$

where Wᵢ and VBIᵢ are the weight percentage and viscosity blending index of each component, respectively. Finally, the kinetic viscosity of the mixture is calculated as:

$${v}_{blend} \approx {e}^{{e}^{\frac{( {VBI}_{blend} - 10.975 )}{14.534}} - 0.8}$$

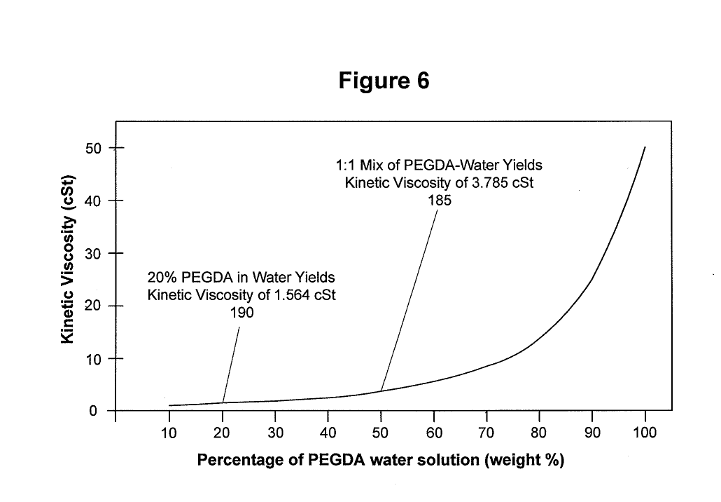

FIG. 6 shows a plot of the estimated kinetic viscosity of an aqueous mixture versus the percentage of PEGDA added as solute. As shown, mixing PEGDA with water in a 1:1 by weight ratio 185 dramatically lowers the kinetic viscosity of the mixture to 3.785 cSt. Moreover, the viscosity of 20% by weight PEGDA 190 in water is estimated to be 1.564 cSt, which is similar to that of water. For this reason, the proof-of-concept experiments described herein were conducted using 20% by weight PEGDA in deionized water. Adequate viscosity and Reynolds numbers are also obtained using 1.2 to 2.5% PNIPAMM aqueous solutions.

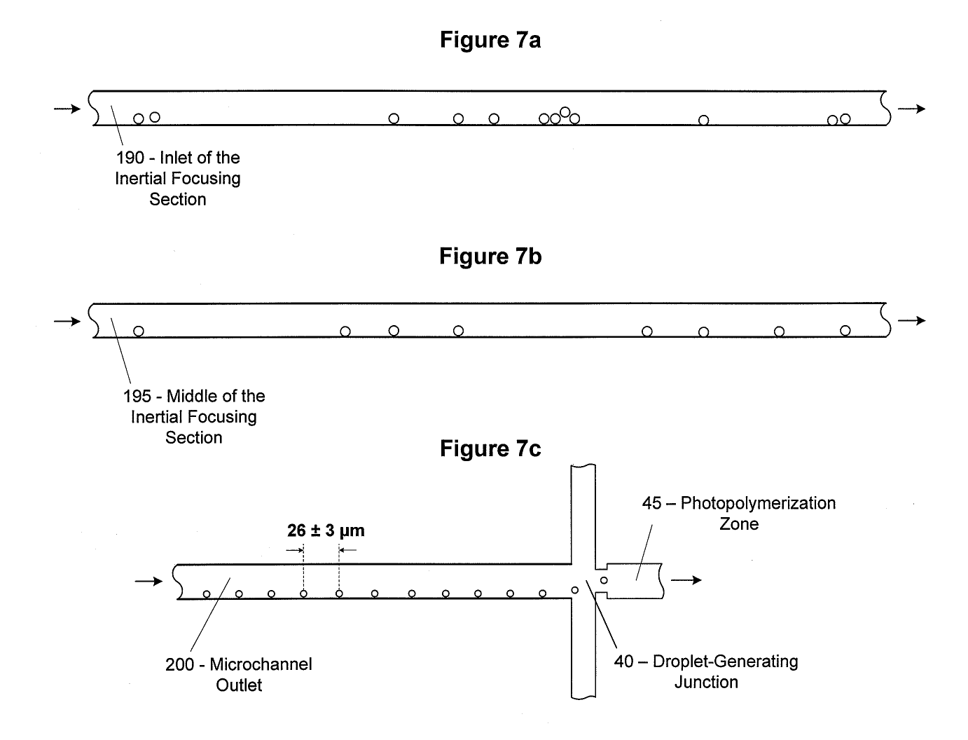

Inertial focusing of the 10.2 μm polystyrene beads was demonstrated using both 20% PEGDA and 1.2-2.5% PNIPAMM aqueous solutions and straight-channel microfluidic devices of the present invention, including the embodiment depicted in FIG. 5a. In this embodiment, inertial focusing is observed for prepolymer flow rates ranging from about 8 to 22 μL/min and corresponding oil flow rates ranging from about 50 to 80 μL/min. FIGS. 7a-c shows images taken at the inlet 190, middle 195, and outlet 200 portions of the inertial-focusing region of the microchannel, tested at a flow rate of 8 μL/min. As shown in FIG. 7a, at the inlet 190 the beads were not uniformly distributed. FIG. 7b shows the beads in the middle of the inertial focusing section 195, where they have become more focused. Finally in FIG. 7c, at the outlet 200 of the channel, the beads have attained a regular order, with a center-to-center separation of 26±3 μm just prior to the droplet-generating junction 40. Similar results were obtained for all flow rates between 8-22 uL/min. The rate of microcapsule 80 formation may exceed 200 Hz.

Proper droplet formation depends upon maintaining certain parameters of liquid viscosity, velocity and surface or interfacial tension between the hydrogel and the oil layers. These parameters are embodied with the capillary number as follows:

$${C}_{a} = \frac{μ V}{γ}$$

where μ is the viscosity of the liquid, V is a characteristic velocity and γ is the surface or interfacial tension between the two fluid phases. Typically, lowering the capillary number less than 1 will increase the chance of “dripping,” which yields monodispersed microcapsules, as opposed to undesirable “jetting,” which may yield microcapsules of variant size. In practice, there is very limited freedom in varying liquid viscosity (μ) and surface or interfacial tension (γ) due to the material choice. However, liquid velocity (i.e., flowrate, V) can be lowered by increasing the volume of the microchannel immediately after the droplet-generating junction 40.

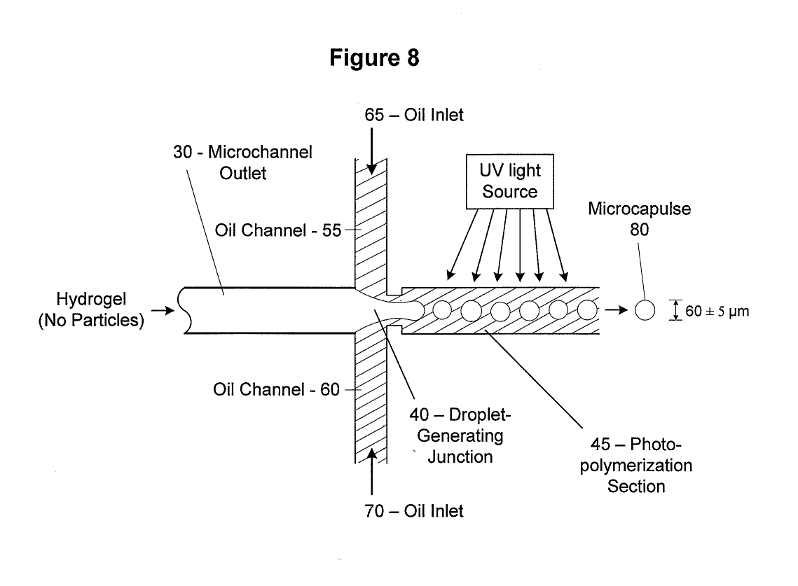

Droplet generation and photo-polymerization to form monodisperse microcapsules was demonstrated using both the linear (straight) and curved (spiral)-channel embodiments of the present invention. FIG. 8 illustrates the process of forming monodisperse PEGDA microcapsules measuring 60±5 μm in diameter. Using the embodiment depicted in FIG. 5a, the droplet-generation rate was observed to be greater than 200 Hz using an oil flowrate from about 50-60 μL/min and a hydrogel flowrate from about 8 to 20 μL/min.

In general, the encapsulation material can be any desirable biocompatible prepolymer with a viscosity close to that of water. Higher viscosities will increase the minimum flow rate needed for inertial focusing in a given channel, which will increase the pressure on the channel wall possibly leading to failure of the device. We have tested UV-curable PEGDA and thermally curable PNIPAAM successfully using both straight-channel and curved-channel devices of the present invention. However, the present invention is not limited to the use of these prepolymer bases. In a typical experiment using the straight-channel device depicted in FIG. 5a, 10 μm particles were shown to undergo inertial focusing and polymerization at flowrates from about 8 to 14 μL/min (Re2.9-5.2) using both 20% PEGDA (0.3-1% Irgacure 2959, 365 nm at 400-1000 mJ/cm², depending on the ambient oxygen concentration) and 1.2-2.5% PNIPAMM (temperature greater than 32° C.) suspensions. The volume fraction (φ) of the particles may be in the range from 1% to 6% depending on the channel geometry, preferably greater than 1.8%. A typical prepolymer suspension (hydrogel) 95 is, for example, prepared by dissolving 20% (w %) of poly(ethyleneglycol)-diacrylate (PEGDA, Mn 575, Sigma Aldrich) in deionized water, then adding the polystyrene beads and a stabilizing agent (1% Tween 20, Sigma Aldrich) under adequate mixing to produce a homogenous mixture. Irgacure 2959 (Ciba), a photoinitiator, is then added to the suspension in a 1% w/w ratio. Fluorinert oil (FC-40, 3M) mixed with 2% biocompatible surfactant (Raindance Tech) is typically, but not exclusively, used as the continuous phase immiscible with the prepolymer mixture.

Another aspect of the present invention is a method and device employing a curved (spiral) inertial-focusing section 140, which provides for a more compact device capable of continuously, and reproducibly, separating (sorting) and microencapsulating individual particles of different sizes from mixtures of particles. In a curved (spiral) channel the addition of curvature introduces a secondary cross-sectional flow field perpendicular to the flow direction, which is known as the Dean flow. It is known that particle trains in curved channels can be consolidated into a single train under the balance of inertial forces and the Dean force, F_{D}, such that the equilibrium position of the particles changes with variations in both the Reynolds number (Re) and the Dean Number (De). The Dean Number depends on the Reynolds number as follows:

$${D}_{e} = {{R}_{e} ( \frac{a}{2 r} )}^{\frac{1}{2}}$$

where Rₑ is the Reynolds number, a is the particle diameter, and r is the curvature of the channel loop. The Dean force is dependent upon the fluid mean velocity and curvature of the channel loop as follows:

$${F}_{D} \sim \frac{ρ {U}_{m}^{2} {aD}_{h}^{2}}{r}$$

$${D}_{h} = \frac{{}_{2}^{}{w}{}_{h}^{}}{( w + h )}$$

where ρ is the fluid density, Uₘ is the fluid mean velocity, r is the curvature of the channel loop, and the hydraulic diameter of the channel, Dₕ, depends on the width, w, and height, h, of the channel. The presence of the Dean force generates a double-recirculating vortex, such that under certain conditions particles of different sizes in a spiral channel can migrate across the flow to equilibrium positions that vary based on the particle sizes.

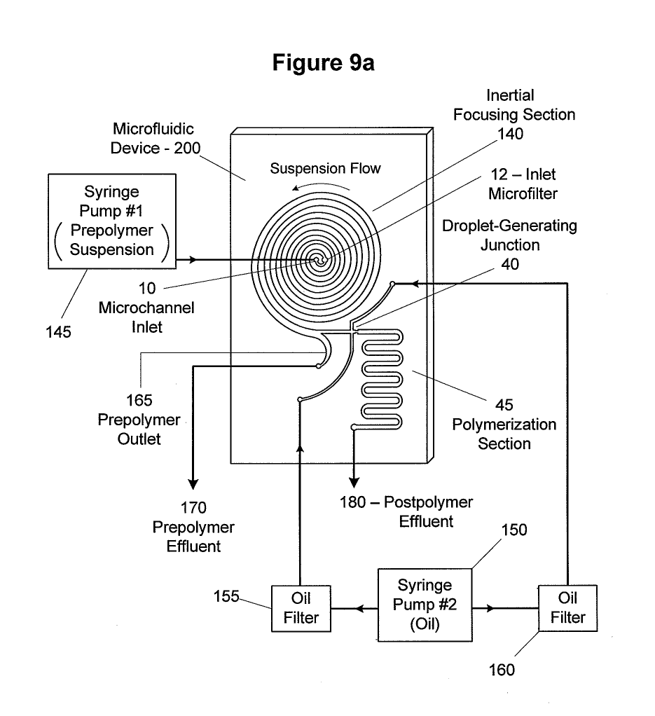

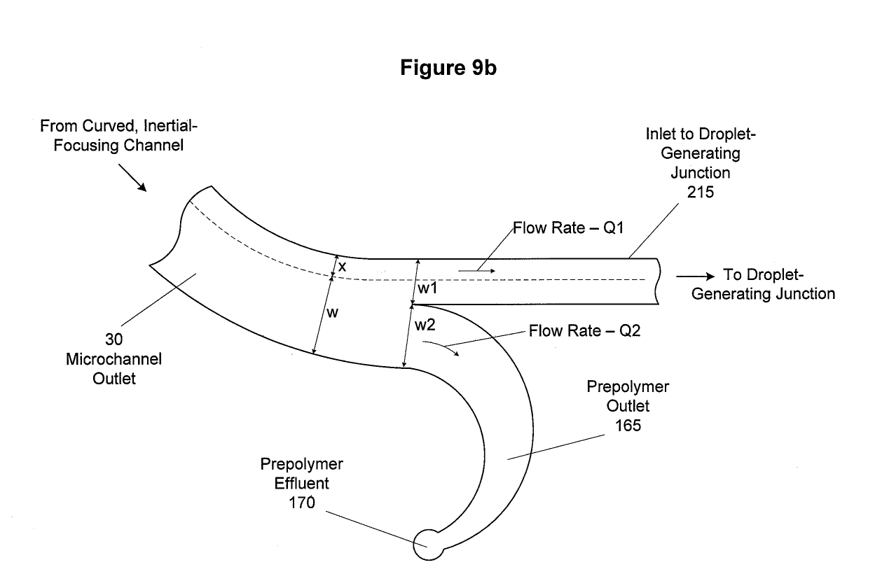

FIG. 9a illustrates the experimental setup for one embodiment of a curved-(spiral)-channel microfluidic device 200 capable of sorting, focusing and encapsulation. This embodiment reduces the footprint of the linear-(straight)-channel device 135 depicted in FIG. 5a (14 cm²) to 6 cm². The curved-channel microfluidic device 200 comprises the same general components as the linear (straight) embodiment depicted in FIG. 5a, except that the inertial-focusing section 140 is curved (spiral) and a prepolymer outlet 165 exists to allow removal of prepolymer effluent 170, and preventing certain particles from entering the droplet-generating junction 40. In one embodiment, the inertial-focusing section 140 is comprised of 8 spiral turns with increasing radius (1.68 mm to 9.46 mm) and channel width (250 μm to 1100 μm). In other embodiments exhibiting comparable results, the channel width of 250 μm is constant and the radius increases from 1.7 mm to 5.8 mm. In still other embodiments, the number of spiral turns may be increased or decreased with corresponding increases or decreases in the radius, and the channel width may be held constant or increased from about 50 μm to 2000 μm, preferably 250 μm to 1100 μM. An inlet microfilter 12 (see FIG. 5b) is positioned downstream of the microchannel inlet 10 to eliminate clumps that may block the junction 40. In some embodiments, one or more prepolymer outlet 165 may be used at the end of the inertial focusing section 140 to ensure removal of excess hydrogel and particles. The droplet-generating junction 40 and polymerization section 45 function identically to those of the straight-channel embodiments described above (see FIG. 5a). In the experiment depicted in FIG. 9a, the hydrogel prepolymer suspension 95 and the oil phase are driven at different flow rates by two separate syringe pumps 145 and 150 (KDS-210, KD Scientific).

In embodiments using UV-initiated polymerization, 20% PEGDA (0.3-1% Iracure 2959) is polymerized by 365 nm photons at 400-1000 mJ/cm² (depending on the ambient oxygen concentration), which is generated by a UV light source (LC8, Hamamatsu). In embodiments using thermal-initiated polymerization, 1.25% PNIPAMM is polymerized at temperatures exceeding 32° C.

Using the embodiment depicted in FIG. 9a, 10 μm particles may be inertially focused using prepolymer flow rates from about 0.7 to 1.0 mL/min (Re=14.7 to 20.9). Separation (sorting) and focusing of 20 μm from 10 μm particles can be successfully performed when the estimated Reynolds number ranges from about 63 to 94 (3 mL/min to 4.5 mL/min). Proper sorting and microdroplet generation using curved-(spiral)-channel embodiments of the present invention relies, in part, upon maintaining certain design parameters for the prepolymer outlet 165 and the inlet 215 to the droplet-generating junction 40. FIG. 9b illustrates the ideal arrangement and dimensions for the inlet 215 to the droplet-generating junction 40 and the prepolymer outlet 165 (referred to cumulatively as “outlets”). The dashed line represents the streamline flow of focused particles. Typically, if there are n outlets 165 with widths:

w_{i-1.n}=a_{i-1.n}

the flow rate, Qₘ, in the mth outlet is given by:

$${Q}_{m} = Q \times \frac{{a}_{m}^{2}}{{\sum }_{i = 1}^{n} {a}_{i}^{2}}$$

where the oil flow rate is typically 4-7 times that of the prepolymer suspension flow rate. Therefore, if the equilibrium position (where x is the distance to the inner wall and w is the remaining channel width) is b:

$$\frac{x}{w} = b$$

and there are two branched outlets with the inner to outer channel-width ratio:

$$\frac{w 1}{w 2} = c$$

then to ensure that particles go to outlet w1, c has to satisfy the following parameters:

$$\frac{{Q}_{1}}{{Q}_{2}} = \frac{{c}^{2}}{1 + {c}^{2}} > b or c > \sqrt{\frac{b}{1 - b}}$$

and vice versa.

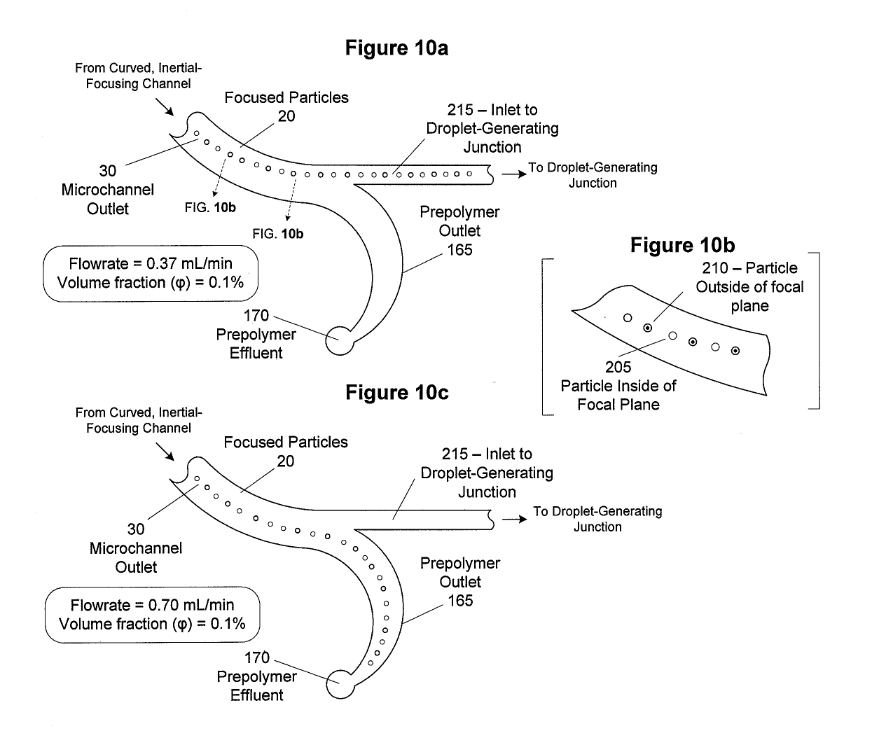

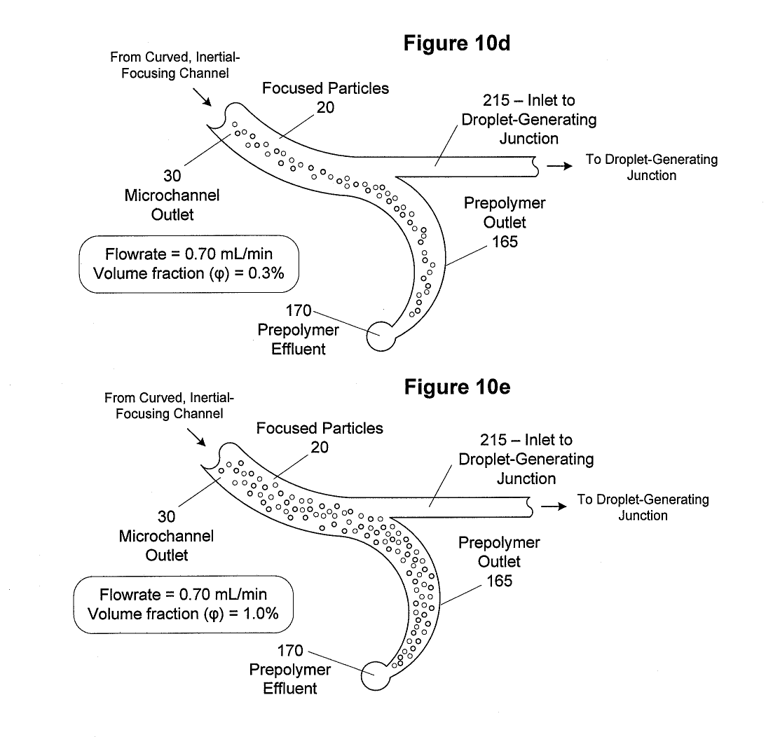

FIGS. 10a-10e illustrate the results obtained using the curved-(spiral)-channel device depicted in FIG. 9a, and sorting/encapsulating polystyrene particles of different sizes. FIG. 10a illustrates the particle flow at the outlet 30 of the curved inertial-focusing channel device 200 at a flowrate of 0.37 mL/min and with particle loading corresponding to volume fraction (φ) of 0.1%. At a flow rate of 0.37 mL/min, the particles 20 start to form an evenly spaced streamline with a staggered pattern (see FIGS. 2a and 2b). FIG. 10b shows an exploded view of the microchannel outlet 30 showing focused and staggered particles 20 observed both inside 205 and outside 210 of the focal plane.

The width of the streamline is directly related to the volume fraction of the particle suspension. In FIG. 10d, for example, increasing the volume fraction from 0.1% to 0.3% (compare FIG. 10c to FIG. 10d) causes significant broadening of the width of the streamline. In FIG. 10e, increasing the volume fraction from 0.3% to 1.0% further increases the width of the streamline. In most instances, the volume fraction (φ) of the prepolymer suspension 95 acts as a stronger limiting factor to control the width of the streamline than does its flow rate.

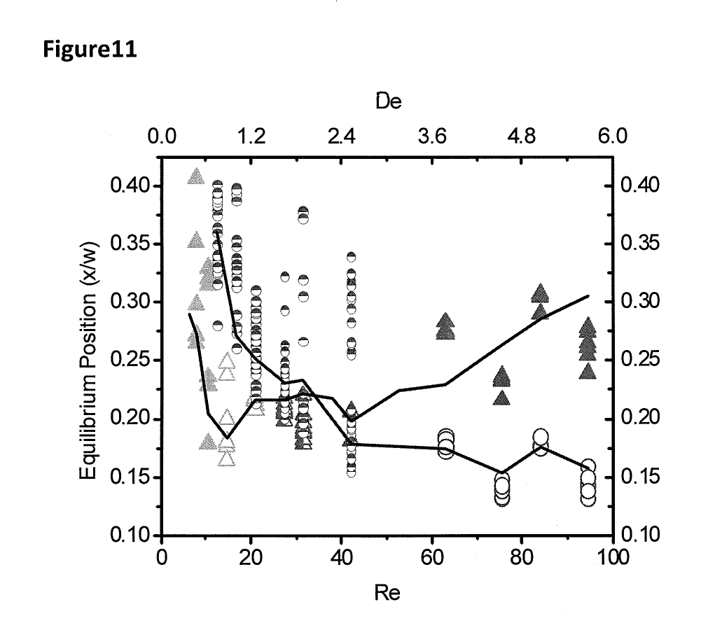

Using the microfluidic device 200 depicted in FIG. 9, with hydrogel flowrate at 0.3 mL/min and volume fraction fixed at 0.1%, microcapsules containing single 20 μm polystyrene beads were selectively produced from mixtures containing 10 μm and 20 μm polystyrene. Modulating the estimated Reynolds number for the prepolymer suspensions 95 used in these experiments revealed a particle-dependent relationship affecting inertial focusing and particle sorting. FIG. 11 shows an equilibrium position study of two particle sizes under different Reynolds number. The solid-line upper curve and solid-line lower curve represent the group behavior of the equilibrium positions (highest probability) as a function of Re for the 10 μm beads and the 20 μm beads respectively, on which curves each data point is the intensity peak acquired by plotting the intensity profile of a composite image overlaid with 500 to 1000 snapshots. The scattered symbols represent equilibrium positions of the 10 μm beads and 20 μm beads measured by random sampling each snapshot. The filled triangles represent multiple 10 μm beads trains coexisting in the flow. The hollowed triangle represents single 10 μm beads trains. The half filled triangles correspond to the twisted 10 μm beads trains and the filled triangles correspond to the fully mixed 10 μm beads (“unfocused”). The half filled circles represent multiple 20 μm beads trains coexisting in the channel. The hollowed circles represent single 20 μm beads trains. The study shows that separation of 10 μm and 20 μm particles can happen in two Re regions (7.8-20.9, 63-94.5), while within an intermediate Re (20.9-42) the two particles have overlapped equilibrium positions. Selective encapsulation of 10 μm particles from mixtures of 10 μm and 20 μm particles may occur when the Reynolds number ranges from about 7.8 to 20.9. Mixtures of inertially-focused 10 μm and 20 μm particles are observed when the Reynolds number ranges from about 21 to 42. Complete separation and encapsulation of 20 μm particles from 10 μm particles occurs at Reynolds numbers above 63, permitting selective microencapsulation of 20 μm particles from mixtures of 10 μm and 20 μm beads.

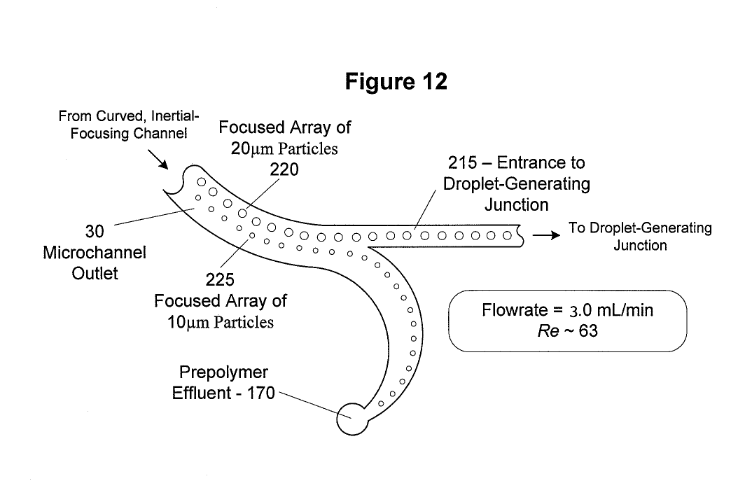

FIG. 12 illustrates the selective encapsulation of 20 μm beads from 10 μm beads using the curved-(spiral)-channel device 200 depicted in FIG. 9. These results obtain at a prepolymer suspension 95 flow rate of 3.0 mL/min and a Reynolds number of 63. As shown, the focused array of 20 μm particles 220 forms a streamline having an equilibrium lateral position significantly lower than that of the 10 μm particles 225, such that only the 20 μm particles enter the entrance to the droplet-generating junction 215.

Another aspect of the invention is a method for continuously generating microcapsules of controlled occupancy and size, wherein functional “tags” and/or “handles” may be added to the microcapsules during microencapsulation to allow easy detection and physical manipulation. The ability to add additional ingredients to microcapsules generated using the devices described herein permits incorporation of functional characteristics, such as fluorescence, magnetism, quantum dots and other features useful for manipulation, monitoring and measurement. The addition of the tags can add functionality to the capsules. For example, fluorescent tags and quantum dots can help visualizing the capsules, and magnetic particles can facilitate magnetic imaging (MRI) and magnetic manipulation of the capsules. Using existing technology, such tags and handles are currently added to cells by modifying the cell surface biochemically. The present invention, however, avoids the need to devise complex chemical strategies often requiring extensive experimentation to implement.

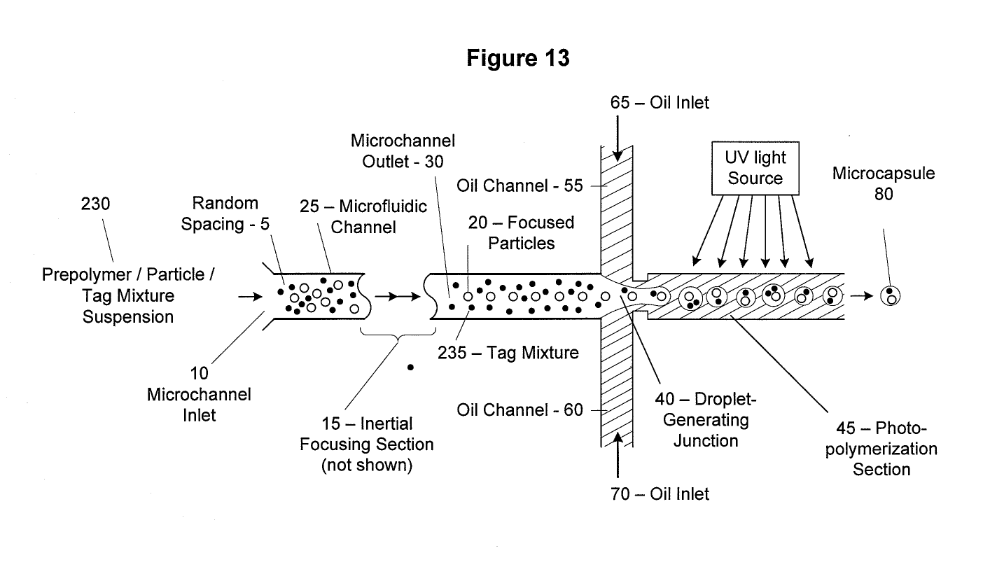

FIG. 13 illustrates the use of one embodiment of the present invention to incorporate a “tag” (micro- or nano-particles) into a particle-containing microcapsule 80. As shown, a prepolymer-tag mixture (suspension) 230 is introduced into a linear or curved-channel device and the resulting focused particles 20 to be encapsulated are mixed with the tag mixture 235, which does not undergo inertial focusing due to their significantly smaller sizes. During droplet generation the tags are incorporated into the microdroplets at a fixed and predictable concentration directly related to the concentration within the prepolymer-tag mixture 230. The microcapsule is formed incorporating the tag. We have demonstrated the manipulation of a magnetically-tagged microcapsule (encapsulating iron oxide superparamagnetic micro particles), which was produced using this device, by using an external permanent magnet. Provided that the added ingredient(s) are more soluble in the prepolymer suspension 230 than the oil layer, they will remain in the microdroplets and become frozen into the microcapsule.

The novel methods and devices described herein may be applied to a wide range of applications besides cell therapeutics. For example, in the materials sciences the delivery and monitoring of nanodevices to parts of the body could facilitate the study and use of man-made tools for treating, studying and monitoring the body. In pharmaceuticals, proper dosing and selective targeting can be facilitated by encapsulating therapeutics within porous microcapsules placed in certain parts of the body. Other therapies involving the use of sub-cellular bioparticles, such as proteins, DNA, RNA, etc., can also benefit from selective placement and time release. In the fragrance industry there is a need to encapsulate fragrance components to improve their shelf life and time releasing characteristics.

The above description of certain preferred embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

1. A device for generating microcapsules with particles encapsulated in a polymer comprising:
a microfluidic channel for introducing particles dispersed in a random spacing in a prepolymer suspension fluid, an outlet for exiting particles carried at a relatively even spacing in the suspension fluid, and an inertial-focusing microchannel section between said inlet and said outlet having channel dimensions and shape so as to cause the particles to become relatively evenly spaced within a streamline flow of the suspension fluid exiting said outlet;
a droplet-generating junction arranged in communication with said outlet of said microchannel and having two opposing oil channels for introducing an oil phase fluid evenly on opposing sides of the flow of particles passing through said junction so as to create separate droplets of prepolymer suspension fluid encapsulating respective particles in the streamline flow; and
a polymerization section for exposing the droplets to a physical force causing polymerization of the prepolymer suspension fluid so as to form a polymer coating on separate microcapsules each containing a respective particle.

2. The device of claim 1, wherein the said inertial-focusing microchannel section is a linear microchannel section.

3. The device of claim 2, wherein said linear microchannel section has a length in the range of 4 cm to 15 cm, preferably 6 cm, a width in the range of 10 μm to 50 μm, preferably 27 μm, and a height of 20 μm to 100 μm, preferably 50 μm.

4. The device of claim 2, wherein said linear microchannel section is dimensioned and shaped for encapsulating particles in the range of about 10 μm diameter particles within droplets in the range of about 60 μm droplets at a rate greater than 200 Hz.

5. The device of claim 1, wherein said inertial-focusing microchannel section is a curved microchannel section.

6. The device of claim 5, wherein said curved microchannel section is comprised of between 5 and 20 spiral turns, preferably 8 spiral turns, of increasing radii from about 1.5 mm to about 25 mm, preferably from about 1.68 mm to 9.46 mm.

7. The device of claim 5, wherein said curved microchannel section is dimensioned and shaped for microencapsulating particles in a range of about 7 μm to 100 μm diameter at a rate of greater than 200 Hz.

8. The device of claim 1, wherein said polymerization section exposes the droplets to one of the physical forces of UV light and heat to initiate polymerization to form the microcapsules.

9. The device of claim 1, wherein the prepolymer suspension fluid is an aqueous solution of a biocompatible prepolymer hydrogel with a viscosity close to that of water.

10. The device of claim 1, wherein the prepolymer suspension fluid is an aqueous solution of poly(ethylene-glycol)-diacrylate (PEGDA) of a concentration in the range of about 10% to 50% (w/w), preferably 20% (w/w).

11. The device of claim 1, wherein the prepolymer suspension fluid is an aqueous solution of poly(N-isopropyl-acryamide) (PNIPAMM) of a concentration in the range of about 0.5% to 5.0% (w/w), preferably 1.2% to 2.5% (w/w).

12. A method for generating microcapsules with particles encapsulated in a polymer coating comprising:
introducing particles in a random spacing in a prepolymer suspension fluid into an inertial-focusing microchannel for causing the particles to become relatively evenly spaced within a streamline flow of the suspension fluid;
providing a droplet-generating junction in communication with the microchannel and having at least two opposing oil channels for introducing an oil phase fluid evenly on opposing sides of the flow of particles passing through the junction so as to create separate droplets of prepolymer suspension fluid encapsulating respective particles in the streamline flow; and
exposing the droplets to a physical energy causing polymerization or gelation of the prepolymer suspension fluid so as to form a polymer coating on separate microcapsules each containing a respective particle.

13. A method according to claim 12, wherein said exposing of the droplets is to one of the physical energies of UV light and heat to initiate solidification to form the microcapsules.

14. A method according to claim 12, further comprising the step of adding a material to the prepolymer suspension fluid having a property of providing a functional tag or handle to the resulting encapsulated microcapsules.

15. A method according to claim 12, wherein the particles are encapsulated to form microcapsules for use in an application selected from the group consisting of cell therapeutics; delivery of nanodevices in the body; dosing of pharmaceuticals in the body; targeting therapeutics in the body; delivery of sub-cellular bioparticles in the body, such as proteins, DNA and RNA; and encapsulating fragrance components to improve shelf life and time releasing characteristics.
